(19) United States
(12) Patent Application Publication
(10) Pub. No.: US 2003/0229012 A1
(43) Pub. Date: Dec. 11, 2003

(54) Human transferring for the treatment of anemia of chronic disease (ACD) and of functional iron deficiency

(76) Inventor: Lothar Thomas, Frankfurt (DE)

Correspondence Address: Finnegan, Henderson, Farabow,, Garrett & Dunner, L.L.P., 1300 I Street, N.W., Washington, DC, 20005-3315, US

(21) Appl. No.: 10/405,612

(22) Filed: Apr. 3, 2003

(30) Foreign Application Priority Data
Apr. 5, 2002 (DE) 10215315.9

(51) Int. Cl. A61K 38/40

(52) U.S. Cl. 514/6

(57) ABSTRACT

A pharmaceutical preparation comprising human transferrin for the treatment of anemic diseases and particularly anemia of chronic disease (ACD) and of functional iron deficiency is described. The human transferrin can be either iron-free or iron-loaded and also used in combination with erythropoietin. A method for the treatment of anemic diseases by administering human transferrin is also described.

### DESCRIPTION OF THE INVENTION

The invention relates to the use of human transferrin alone or in combination with iron or in combination with erythropoietin or in combination with iron and erythropoietin for the treatment of anemia of chronic disease (ACD) and of functional iron deficiency.

1. Human transferrin

Human transferrin is a protein with a molecular weight of 88 kD and is synthesized in the liver. Besides the protein portion it has carbohydrate chains which account for about 10% of the molecular weight. Iron-free transferrin (apotransferrin) binds two atoms of 3-valent iron. Normally from 16 to 45% of apotransferrin molecules are loaded with iron. The transferrin concentration in the blood is responsible for the iron distribution in the body. Transferrin transports the iron from the iron-storage tissues into the bone marrow for hemoglobin synthesis. Hemoglobin released by aging blood cells delivers its iron back to transferrin. Transferrin deposits this in iron-storage tissues and collects it from there again when there is a need in the bone marrow. Transferrin is thus, in global terms, responsible for the iron turnover in the body.

The concentration of transferrin in blood serum is normally from 2.0 to 3.5 g/l. In total body iron deficiency, the liver produces more transferrin, and the concentration in the serum is increased since the turnover of the iron must be increased in order to supply the bone marrow. The iron turnover is reduced in ACD. The reason for this is the diminished transferrin synthesis during chronic infections, inflammatory disorders and cancers, since the liver downregulates the transferrin synthesis in order to be able to ensure increased synthesis of defense proteins (acute-phase proteins). As a consequence, there is a decrease in the transferrin concentration in the blood and thus also in the iron turnover and the hemoglobin production in the erythropoietic precursor cells of the bone marrow. It is also known that an altered transferrin is produced during inflammatory disorders. Thus, transferrins with more highly branched glycan side chains have been described in rheumatoid arthritis (Feelders G. A. et al., Rheumat Int, 12: 195 to 199 (1992)). It is to be assumed that transferrin is able to deliver less iron to the transferrin receptors of hemoglobin-producing erythropoietic precursor cells when it has altered glycan side chains or is present in diminished concentration.

2. Area of application

About 10% of the population of industrialized countries has anemia (deficiency of blood). The most common cause of anemia is inadequate availability of iron, which is the precondition for the production of the red blood pigment hemoglobin in the erythropoietic precursor cells of the bone marrow. Hemoglobin is the main constituent of red blood cells, which transport oxygen from the lung into the tissues and CO₂ from the tissues into the lung. The inadequate availability of iron for hemoglobin production is caused either by a reduction in the total body iron or an iron distribution impairment. Iron-deficiency anemia is present in the first case, while in the second the amount of total body iron is normal or even increased but is provided only inadequately to the bone marrow for hemoglobin production.

This state, in which the total body iron is adequate but the requirements for hemoglobin production is defective, is referred to as functional iron deficiency. This is the case in all anemias occurring during chronic infections, inflammatory disorders and cancers (Means RT, Krantz SB, Blood, 80: 1639 to 1647 (1992)). This is also the case in all anemias with inadequately low erythropoietin production where, after stimulation of red blood cell production by recombinant human erythropoietin (r-HuEPO), the supply of iron to the bone marrow is inadequate. This is the classic situation in the treatment of dialysis patients in the final stage of chronic renal failure with r-HuEPO. This is because therapeutic administration of r-HuEPO is able to stimulate red blood cell production to such an extent that the requirement for iron by the proliferating bone marrow may far exceed the supply (Cavill 1, Blood, 83: 1377 (1993)).

3. Use of human transferrin

Human transferrin-containing pharmaceutical preparations have already found therapeutic uses. Thus, U.S. Pat. Nos. 6,251,860 and 6,326,473 disclose the use of preparations containing apotransferrin, i.e. the iron-free form of transferrin, for binding non-transferrin-bound iron (NTBI) which occurs during cytotoxic therapy in cancer patients and may lead to tissue damage. The use of iron-free or iron-containing human transferrin for the treatment of anemias, especially of ACD and of functional iron deficiency is, however, not considered or suggested therein.

Adequate supply of iron to the bone marrow has become increasingly important in recent years because, owing to the increasing use of r-HuEPO in patients suffering from anemia and the hemoglobin production stimulated thereby, an increasing depletion of iron from the bone marrow is observed, and this can no longer be adequately controlled by iron-replacement therapies disclosed to date. Additional administration of iron in r-HuEPO therapy leads in these cases to a multiplication in total body iron and damage to organs through oxidative stress.

It has now been found, surprisingly, that these problems can be solved in a satisfactory way through administration of human transferrin.

The invention therefore relates to the use of a preparation which comprises a therapeutically effective amount of human transferrin and is suitable for parenteral administration for the treatment of ACD and of functional iron deficiency. This preparation may comprise iron-free or iron-loaded human transferrin, depending on the patient's iron status.

Particularly good therapeutic results are achieved in the treatment of anemic disorders with a combination product which comprises, separate from one another or present in a mixture, preparations suitable for parenteral administration of therapeutic amounts of an

a) iron-free or iron-loaded human transferrin and/or

b) erythropoietin.

The human transferrin employed for this can be obtained from serum or plasma. It is likewise possible to employ a recombinant transferrin, in which case correct glycosylation is important. A suitable method for obtaining iron-free human transferrin is disclosed in European Patent Application 0 490 384, which is incorporated herein by reference. However, it is preferred to use human transferrin saturated with iron.

The erythropoietin employed for the pharmaceutical preparations of the invention can be produced by recombinant methods.

It has now been possible to show unambiguously that administration of purified human transferrin has a beneficial effect in the treatment of ACD and in functional iron deficiency. Within 5 days there is an increase in the hemoglobin in the erythropoietic precursor cells. In the case of ACD, the transferrin concentration in the serum is reduced (<2 g/l) and the transferrin is structurally altered in the carbohydrate side chains. In addition, the saturation of the transferrin with iron is diminished (transferrin saturation <16%). In functional iron deficiency, the iron turnover is inadequate since the proliferating erythropoiesis is not adequately supplied with iron by iron-loaded transferrin.

Surprisingly, intravenous administration of apotransferrin (iron-free transferring, transferrin or iron-saturated transferrin now improves the iron supply for erythropoiesis and thus corrects an anemia. The administration of the particular preparation depends on the patient's total body iron status. Clinical and laboratory diagnostic observations on patients with ACD and functional iron deficiency have shown that administration of fresh frozen plasma containing transferrin with normal iron saturation first increases the hemoglobin content in reticulocytes and then leads to an increase in the hemoglobin level in the blood.

The pharmaceutical transferrin preparations of the invention can be administered as intravenous injection or as infusion. The effective dosage varies from 50 to 250 mg/kg and day and tends toward the lower limit for ACD and toward the upper limit for functional iron deficiency. On administration of iron-saturated transferrin in place of administration of intravenous iron in combination with erythropoietin it was observed that the known side effects of intravenous iron therapies do not occur. Nor do toxic concentrations of non-transferrin-bound iron (NTBI) which are usual during oral iron therapy, occur. The oxidative stress caused by NTBI and the tissue and organ damage associated therewith (Knight J. A. ed. AACC Press, 1 to 392 (1999)) can be prevented by administration of transferrin-bound iron.

What is claimed is:

1. A pharmaceutical preparation for the treatment of anemic disorders comprising a therapeutically effective amount of human transferrin.

2. The pharmaceutical preparation as claimed in claim 1, wherein the human transferrin is obtained from plasma or serum.

3. The pharmaceutical preparation as claimed in claim 1, wherein the human transferrin is recombinantly produced.

4. The pharmaceutical preparation as claimed in claim 1, wherein the human transferrin is a fragment of recombinantly produced human transferrin or a fragment of plasma or serum derived human transferrin.

5. The pharmaceutical preparation as claimed in claim 1, wherein the human transferrin is iron-free or iron-loaded human transferrin.

6. The pharmaceutical preparation as claimed in claim 5, wherein the iron-free or iron-loaded human transferrin is obtained from plasma or serum.

7. The pharmaceutical preparation as claimed in claim 5, wherein the iron-free or iron-loaded human transferrin is recombinantly produced.

8. The pharmaceutical preparation as claimed in claim 5, wherein the iron-free or iron-loaded human transferrin is a fragment of recombinantly produced iron-free or iron-loaded human transferrin or a fragment of plasma or serum derived iron-free or iron-loaded human transferrin.

9. The pharmaceutical preparation as claimed in claim 1 wherein the pharmaceutical preparation is suitable for parenteral, intravenous or infusion administration.

10. A method for the treatment of anemia of chronic disease (ACD) or functional iron deficiency comprising administering the pharmaceutical preparation as claimed in claim 1 to a patient in need thereof.

11. A pharmaceutical preparation for the treatment of anemic disorders comprising a therapeutically effective amount of at least one compound chosen from the group consisting of:
a) iron-free human transferrin;
b) iron-loaded human transferrin; and
c) erythropoietin.

12. The pharmaceutical preparation as claimed in claim 11, wherein the iron-free or iron-loaded human transferrin is obtained from plasma or serum.

13. The pharmaceutical preparation as claimed in claim 11, wherein the iron-free or iron-loaded human transferrin is recombinantly produced.

14. The pharmaceutical preparation as claimed in claim 11, wherein the iron-free or iron-loaded human transferrin is a fragment of recombinantly produced iron-free or iron-loaded human transferrin or a fragment of plasma or serum derived iron-free or iron-loaded human transferrin.

15. The pharmaceutical preparation as claimed in claim 11, wherein the erythropoietin is recombinantly produced.

16. The pharmaceutical preparation as claimed in claim 11 wherein the pharmaceutical preparation is suitable for parenteral, intravenous or infusion administration.

17. A method for the treatment of anemia of chronic disease (ACD) or functional iron deficiency comprising administering the pharmaceutical preparation as claimed in claim 11 to a patient in need thereof.

18. A method for the treatment of anemia of chronic disease (ACD) or functional iron deficiency comprising administering a therapeutically effective amount of iron-loaded human transferrin to a patient in need thereof.

19. The pharmaceutical preparation as claimed in claim 9 wherein the pharmaceutical preparation is administered intravenously.

20. The pharmaceutical preparation as claimed in claim 16 wherein the pharmaceutical preparation is administered intravenously.
